(19) United States
(12) Patent Application Publication
(10) Pub. No.: US 2013/0274794 A1
(43) Pub. Date: Oct. 17, 2013

(54) HEMOSTASIS DEVICE AND METHOD FOR USING THE SAME

(71) Applicant: TD.JAM Medical Technologies, LLC, Ada, MI (US)

(72) Inventors: Nagib Chalfoun, Ada, MI (US); Tony Das, Dallas, TX (US); Jihad Mustapha, Ada, MI (US)

(21) Appl. No.: 13/909,883

(22) Filed: Jun. 4, 2013

Related U.S. Application Data
(62) Division of application No. 12/632,124, filed on Dec. 7, 2009, now abandoned.
(60) Provisional application No. 61/149,190, filed on Feb. 2, 2009.
(60) Provisional application No. 61/120,581, filed on Dec. 8, 2008.

(51) Int. Cl. A61B 17/12

(52) U.S. Cl. CPC A61B 17/12
USPC 606/201

(57) ABSTRACT

A hemostasis device is disclosed. The hemostasis device includes a stem portion including a top end and a bottom end, a handle portion connected to the top end of the stem portion, and a base portion connected to the bottom end of the stem portion. A method for utilizing the hemostasis device is also disclosed.

### PRIORITY CLAIM

This application claims priority to U.S. Provisional Patent Application Ser. No. 61/120,581 filed on Dec. 8, 2008 and U.S. Provisional Patent Application Ser. No. 61/149,190 filed on Feb. 2, 2009, both of which are herein incorporated by reference in their entirety.

### FIELD

The invention relates to a hemostasis device and to a method for using the same.

### BACKGROUND

In some circumstances, it may be desirable to arrest bleeding (i.e. hemostasis) by compressing blood vessels. Referring to FIG. 14, hemostasis may be achieved, for example, by utilizing one's hand, H, and/or digits, D, by directly applying the hand, H, and/or digits, D, to, for example, an abdominal wall, W, of a patient, P. By applying one or more of the hand, H, and digits, D, to the abdominal wall, W of the patient, P, one or more arteries/veins, FA, FV, may be compressed substantially adjacent a bone, B.

While known techniques for achieving hemostasis such as those described above have been proven to be acceptable for various applications, such conventional techniques are nevertheless susceptible to, for example, human error and/or human fatigue such that, if, for example, the hand, H, and/or digits, D, are not properly applied to the patient, P, an asymmetrical pressure to one or more of the arteries/veins, FA, FV, may prevent hemostasis from being achieved. Therefore, a need exists to develop improved technique and a device that advance the art.

### BRIEF DESCRIPTION OF THE DRAWINGS

The present disclosure will now be described, by way of example, with reference to the accompanying drawings, in which:

FIG. 1A is an exploded side view of a hemostasis device in accordance with an exemplary embodiment of the invention;

FIG. 1B is an exploded side view of a hemostasis device in accordance with an exemplary embodiment of the invention;

FIG. 2A is a perspective, assembled view of the hemostasis device of FIG. 1A in accordance with an exemplary embodiment of the invention;

FIG. 2B is a perspective, assembled view of the hemostasis device of FIG. 1B in accordance with an exemplary embodiment of the invention;

FIG. 3A is a cross-sectional view of the hemostasis device of FIG. 2A according to line 3-3 in accordance with an exemplary embodiment of the invention;

FIG. 3B is another view of the hemostasis device of FIG. 3A in accordance with an exemplary embodiment of the invention;

FIG. 4 is a cross-sectional view of a hemostasis device in accordance with an exemplary embodiment of the invention;

FIG. 5 is a cross-sectional view of a hemostasis device in accordance with an exemplary embodiment of the invention;

FIG. 6 is a cross-sectional view of a hemostasis device in accordance with an exemplary embodiment of the invention;

FIG. 7 is a cross-sectional view of a hemostasis device in accordance with an exemplary embodiment of the invention;

FIG. 8 is a cross-sectional view of a hemostasis device in accordance with an exemplary embodiment of the invention;

FIG. 9 is a cross-sectional view of a hemostasis device in accordance with an exemplary embodiment of the invention;

FIG. 10A is a cross-sectional view of a hemostasis device in accordance with an exemplary embodiment of the invention;

FIG. 10B is another view of the hemostasis device of FIG. 10A in accordance with an exemplary embodiment of the invention;

FIG. 11A is a cross-sectional view of a hemostasis device in accordance with an exemplary embodiment of the invention;

FIG. 11B is another view of the hemostasis device of FIG. 11A in accordance with an exemplary embodiment of the invention;

FIG. 12A is a view of a hemostasis device located proximate an abdominal wall of a patient in accordance with an exemplary embodiment of the invention;

FIG. 12B is a view of the hemostasis device according to FIG. 12A located adjacent the abdominal wall of the patient in accordance with an exemplary embodiment of the invention;

FIG. 13 is an enlarged side view of the hemostasis device of FIG. 12B in accordance with an exemplary embodiment of the invention; and

FIG. 14 is a view of a view of a technique for achieving hemostasis with a person's hand and/or digits.

FIG. 15 is an exploded perspective view of a hemostasis device in accordance with an exemplary embodiment of the invention;

FIG. 16 is an assembled perspective view of the hemostasis device of FIG. 15 in accordance with an exemplary embodiment of the invention;

FIG. 17 is a front view of the hemostasis device of FIG. 16 in accordance with an exemplary embodiment of the invention;

FIG. 18 is a side view of the hemostasis device of FIG. 16 in accordance with an exemplary embodiment of the invention;

FIG. 19 is an enlarged perspective view of a portion of the hemostasis device of FIG. 16 in accordance with an exemplary embodiment of the invention;

FIG. 20 is an enlarged perspective view of a portion of a hemostasis device in accordance with an exemplary embodiment of the invention;

FIG. 21 is an enlarged perspective view of a portion of a hemostasis device in accordance with an exemplary embodiment of the invention;

FIG. 22A is a view of a hemostasis device located proximate an abdominal wall of a patient in accordance with an exemplary embodiment of the invention;

FIG. 22B is a view of the hemostasis device according to FIG. 22A located adjacent the abdominal wall of the patient in accordance with an exemplary embodiment of the invention;

FIG. 23 is an enlarged side view of the hemostasis device of FIG. 22B in accordance with an exemplary embodiment of the invention;

FIG. 24 is an enlarged perspective view of a portion of a hemostasis device in accordance with an exemplary embodiment of the invention; and

FIG. 25 is a view of a view of a technique for achieving hemostasis with a person's hand and/or digits.

### DETAILED DESCRIPTION

The Figures illustrate exemplary embodiments of a hemostasis device and method for using the same in accordance with embodiments of the invention, and, based on the foregoing, it is to be generally understood that the nomenclature used herein is simply for convenience and the terms used to describe the invention should be given the broadest meaning by one of ordinary skill in the art.

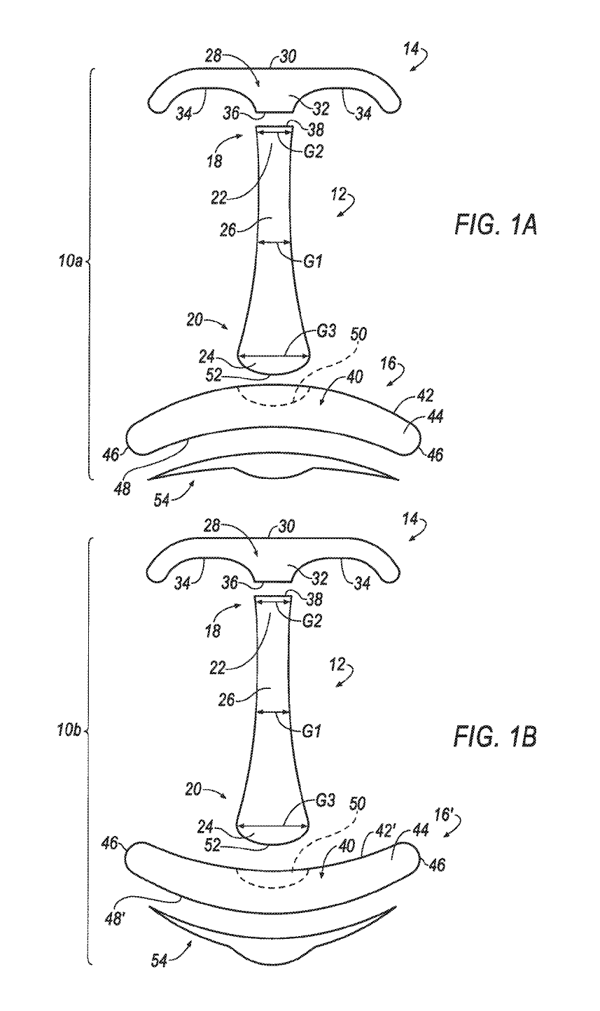

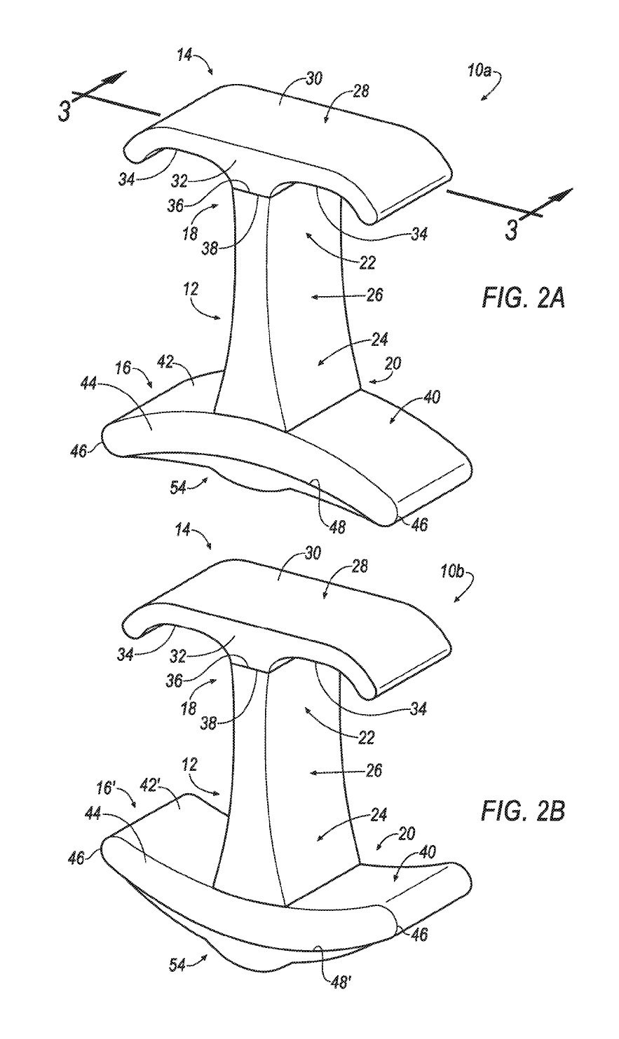

Referring to FIGS. 1A and 2A, a hemostasis device is shown generally at 10a in accordance with an embodiment of the invention. In an embodiment, the hemostasis device 10a may include a centering pole/stem portion 12, a handle portion 14 and a base portion 16. In an embodiment, the stem portion 12 includes a first, top end 18 and a second, bottom end 20. In an embodiment, the handle portion 14 is connected to the stem portion 12 at the first, top end 18, and, the base portion 16 is connected to the stem portion 12 at the second, bottom end 20.

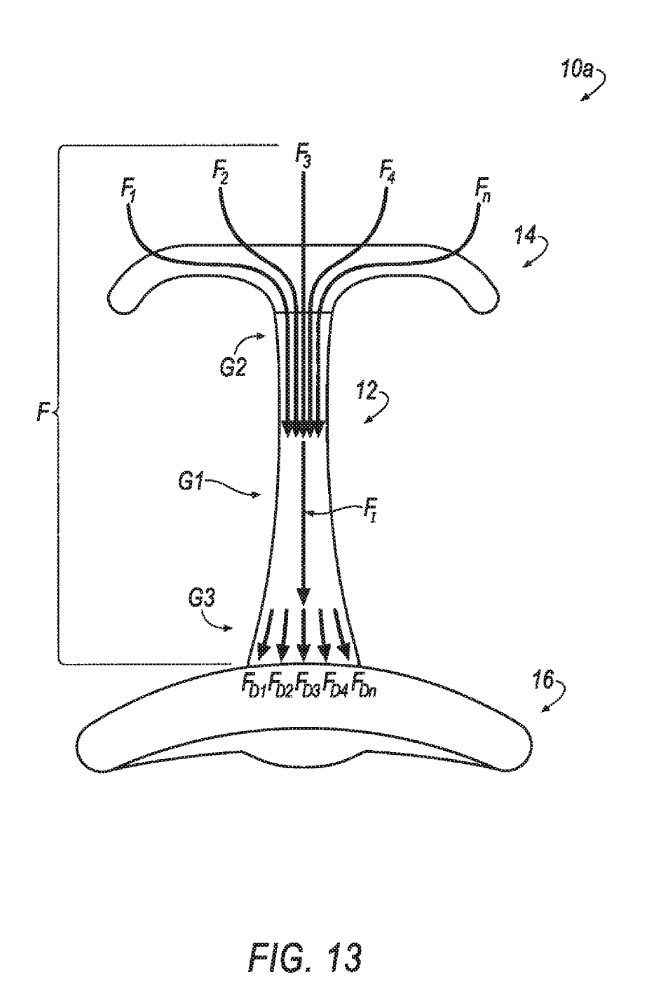

In an embodiment, the stem portion 12 may define a head portion 22 proximate the first, top end 18, a shoulder portion 24 proximate the second, bottom end 20, and a neck portion 26 extending between the head portion 22 and the shoulder portion 24. In an embodiment, the neck portion 26 may define a first, cross-sectional geometry, G1, that is less than a second cross-sectional geometry, G2, defined by the head portion 22, and, in an embodiment, the shoulder portion 24 may define a third cross-sectional geometry, G3, that is greater than the second cross-sectional geometry, G2, defined by the head portion 22. The function of the geometries, G1-G3, of the stem portion 12 are described in greater detail in the foregoing disclosure at, for example, FIG. 13.

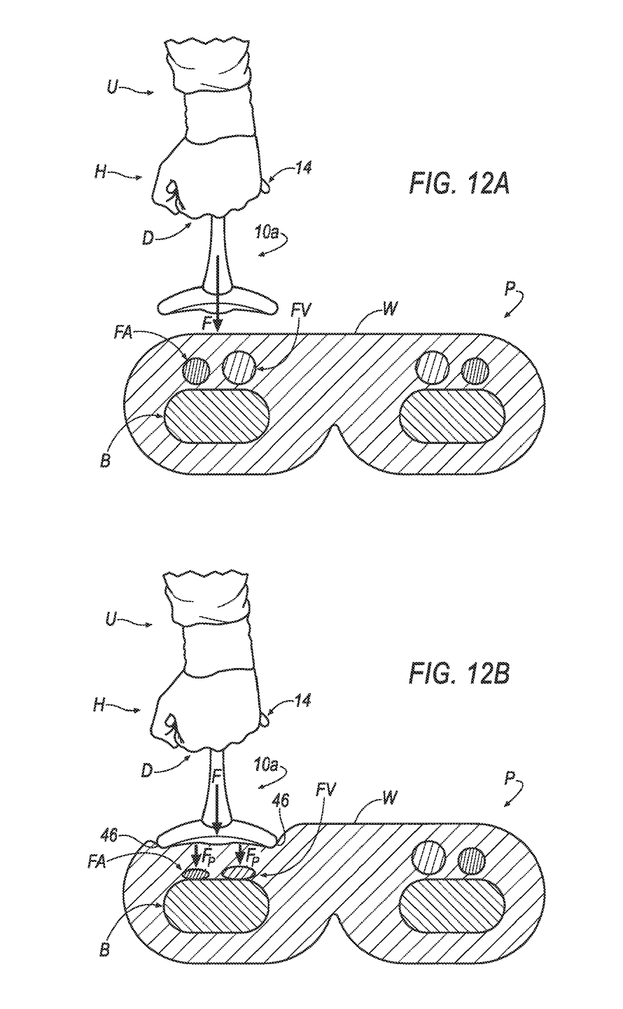

In an embodiment, the handle portion 14 is defined by a body 28 having by an upper, substantially planar surface 30, a side surface 32, a pair of lower, arcuate surfaces 34, and a lower, connection surface 36. In an embodiment, the connection surface 36 permits the handle portion 14 to be connected to an upper connection surface 38 defined by the top end 18 of the stem portion 12. Functionally, in an embodiment as seen in FIGS. 12A-12B, a user, U, may grip the handle portion 14 by locating one's hand, H, substantially adjacent the upper, substantially planar surface 30 and by wrapping one's digits, D, around the side surface 32 and the pair of lower, arcuate surfaces 34.

Referring to FIGS. 1A and 2A, in an embodiment, the base portion 16 is defined by a body 40 having an arcuate, upper surface 42, a side surface 44, arcuate lateral surfaces 46 and an arcuate lower surface 48. In an embodiment, a portion of the arcuate upper surface 42 defines a connection surface, which is shown generally at 50.

In an embodiment, the connection surface 50 may define a recessed cavity formed in the arcuate upper surface 42. In an embodiment, the connection surface 50 permits the base portion 16 to be connected to a lower connection surface 52 defined by the bottom end 20 of the stem portion 12. Further, in an embodiment, the arcuate upper surface 42 and the arcuate lower surface 48 are arranged in a substantially “concave-down” orientation.

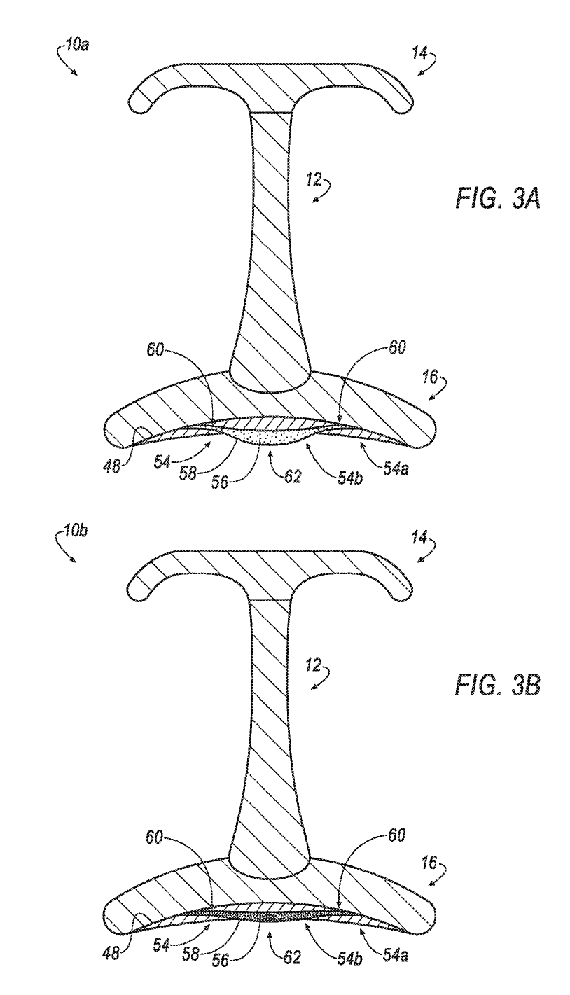

In an embodiment, the hemostasis device 10a may further comprise an optional pad portion 54. In an embodiment, as seen in FIGS. 3A and 3B, the pad portion 54 may include one or more materials, which are shown, generally, at 54a, 54b. In an embodiment, a first material 54a may be defined by a substantially soft consistency with a relatively constant density that is substantially independent to externally-applied forces. In an embodiment, a second material 54b may be defined by a substantially soft consistency with a variable density, which may depend on, for example, an externally-applied force.

In an embodiment, as seen in FIGS. 3A and 3B, the pad portion 54 may be disposed adjacent the arcuate lower surface 48 of the base portion 16. In an embodiment, the second material 54b of the pad portion 54 may be further defined to include a substantially elastic or pliable material 56 disposed by and retained within an enclosed pocket 58 defining substantially narrow, lateral portions 60 and a central, bulged portion 62. In an embodiment, the pocket 58 may be connected to and/or partially encapsulated by the first material 54a such that, for example, the bulged portion 62 extends through a portion of the first material 54a as the substantially narrow, lateral portions 60 are encapsulated/sandwiched by one or more of the first material 54a and the arcuate lower surface 48.

In an embodiment, as seen in FIG. 3A, when the pad portion 54 is in a “relaxed state” (e.g., not applied adjacent a patient, P, as shown in FIG. 12A), the second material 54b of the pad portion 54 may be defined by a substantially soft consistency, having a first density. Conversely, as seen in FIG. 3B, when the second material 54b of the pad portion 54 is in a “non-relaxed state”/“compressed state” (e.g., applied substantially adjacent a patient, P, as shown in FIG. 12B), the second material 54b of the pad portion 54 may be defined by a substantially soft consistency, having a second density. In an embodiment, the second density may be greater than the first density, and, as such, the second density may cause the second material 54b to maintain its soft consistency, yet also appearing to include a substantially rigidified characteristic when compared to that of the relaxed state of the second material 54b shown in FIG. 3A having a lower density.

In an embodiment, the substantially rigidified characteristic of the second material 54b may be achieved by reducing the bulged portion 62 (as a result of, e.g., the hemostasis device 10a being applied substantially adjacent a patent, P) such that the elastic or pliable material 56 proximate the bulge portion 62 is laterally forced toward the narrow, lateral portions 60. In an embodiment, the cross-sectional geometry of the enclosed pocket 58 proximate the narrow, lateral portions 60 may be resistant to an expansion or contraction, and, as such, when the substantially elastic or pliable material 56 proximate the bulged portion 62 is laterally forced toward the narrow, lateral portions 60, the density of the elastic or pliable material 56 at least proximate the bulged portion 62 may be increased such that the second material 54b of the pad portion 54 exhibits the substantially rigid characteristic discussed above.

Referring to FIGS. 1B and 2B, a hemostasis device is shown generally at 10b in accordance with an embodiment of the invention. In an embodiment, the hemostasis device 10b is substantially the same as the hemostasis device 10a with the exception that the base portion, which is shown generally at 16′, includes an arcuate upper surface 42′ and an arcuate lower surface 48′ that are arranged in a substantially “concave-up” orientation. By arranging one or more of the arcuate upper and lowers surface 42′, 48′ in a substantially concave-up orientation, the base portion 16′ may be generally referred to as a “rocker base portion.”

Accordingly, when one or more of the rocker base portion 16′ and the pad portion 54 are disposed adjacent a patient, P, the rocker base portion 16′ permits a user to “rock” the hemostasis device 10b back-and-forth such that a portion of the arcuate lower surface 48′ of the rocker base portion 16′ and pad portion 54 define a variable point of contact with the patient, P. Conversely, referring to FIGS. 1A and 2A, one or more of the arcuate lower surface 48 of the base portion 16 and the pad portion 54 define the hemostasis device 10a having a non-variable point of contact with the patient, P, such that substantially all of the arcuate lower surface 48 of the base portion 16 and/or pad portion 54 is in contact with the patient, P. Further, as seen in FIG. 12B, the rounded shape of the arcuate lateral surfaces 46 of the base portion 12 may also be in contact with the patient, P, such that the arcuate lateral surface 46 do not pinch or “pocket” the skin of the patient, P.

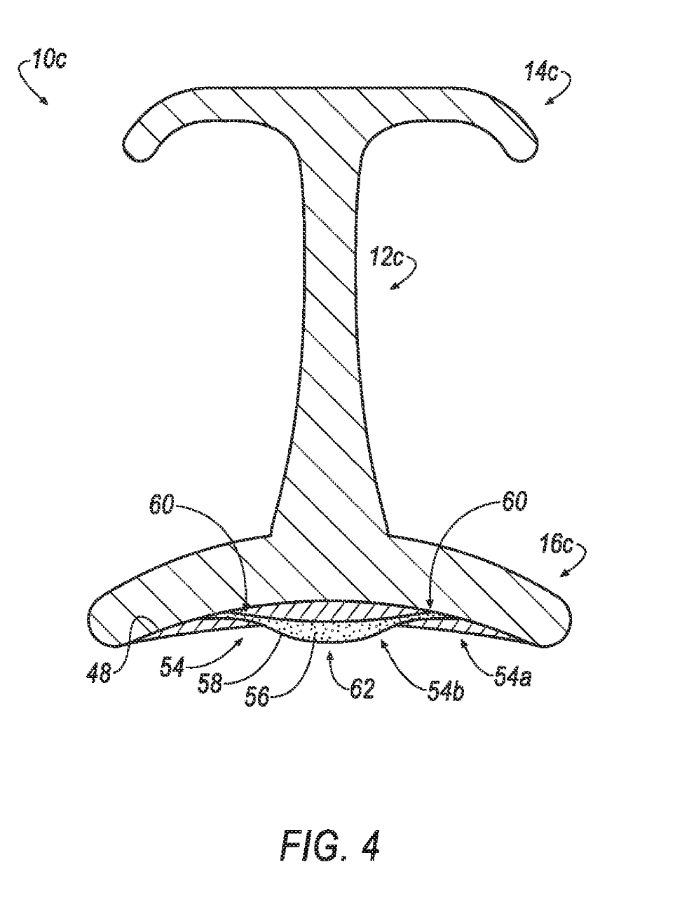

In an embodiment, as seen in FIGS. 3A and 3B, it will be appreciated that although one or more of the hemostasis devices 10a, 10b are described to include separate components such as a handle portion 14 and a base portion 16/16′ each connected to a stem portion 12, the hemostasis devices 10a, 10b are not limited to a plurality of components that are assembled together. Accordingly, referring to, FIG. 4, for example, it will be appreciated that a hemostasis device 10c may include a single, unitary component defined by a stem portion 12c, a handle portion 14c and a base portion 16c. In an embodiment, the hemostasis device 10c may also include the pad portion 54.

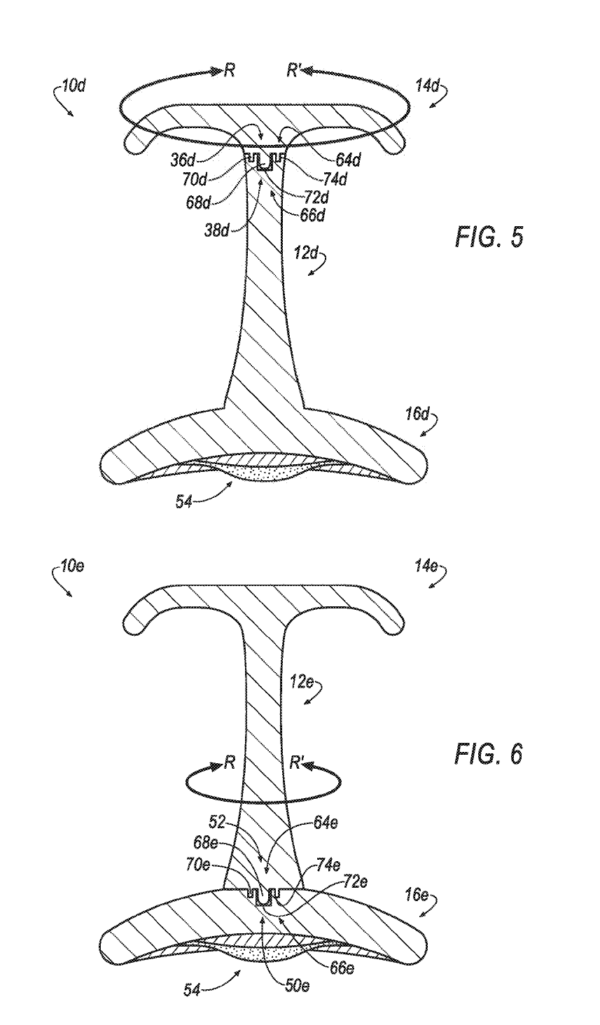

Referring to FIG. 5, an embodiment of the hemostasis device 10d is shown according to an embodiment of the invention. In an embodiment, stem portion 12d and the base portion 16d may define a non-assembled, unitary structure that is connected to the handle portion 14d. In an embodiment, the connection surface 36d of the handle portion 14d may define, for example, a male portion 64d, and, the upper connection surface 38d of the stem portion 12d may define a female portion 66d. In an embodiment, the male portion 64d may be defined to include one or more of a pin 68d, a ring 70d or the like that is disposed into one or more corresponding recesses 72d, 74d defined by the female portion 66d. In an embodiment, the connection of the male portion 64d and the female portion 66d permits the handle 14d to rotate relative the stem portion 12d according to the direction of the arrow, R/R′.

Referring to FIG. 6, an embodiment of a hemostasis device 10e is shown according to an embodiment of the invention. In an embodiment, the stem portion 12e and the handle portion 14e may define a non-assembled, unitary structure that is connected to the base portion 16e. In an embodiment, the lower connection surface 52e of the stem portion 12e may define, for example, a male portion 64e, and, the connection surface 50e of the base portion 16e may define, for example, a female portion 66e. In an embodiment, the male portion 64e may be defined to include one or more of a pin 68e, ring 70e or the like that is disposed into one or more corresponding recesses 72d, 74d defined by the female portion 66e. In an embodiment, the connection of the male portion 64e and the female portion 66e permits the unitary stem portion 12e and handle portion 14e to rotate relative the base portion 16e according to the direction of the arrow, R/R′.

Referring to FIGS. 5 and 6, it will be appreciated that the rotation, R/R′, permits a user, U, to grasp and rotate the handle portion 14d/14e while adjusting his/her positioning relative the patient, P, a full 360°. This permissive adjustment of the position of the user, U, may assist in relieving stiffness/fatigue of the user, U, and/or to make a minor adjustment in the direction of the force vector, F (see, e.g., FIGS. 12A, 12B), to achieve hemostasis. Further, it will be appreciated that although FIGS. 5-6 disclose male and female connections comprising a pin, ring and corresponding recesses, it will be appreciated that the invention is not limited to pins, rings and corresponding recesses for permitting the rotational movement, R/R′, and that any desirable connection may be provided to achieve the rotational movement R/R′.

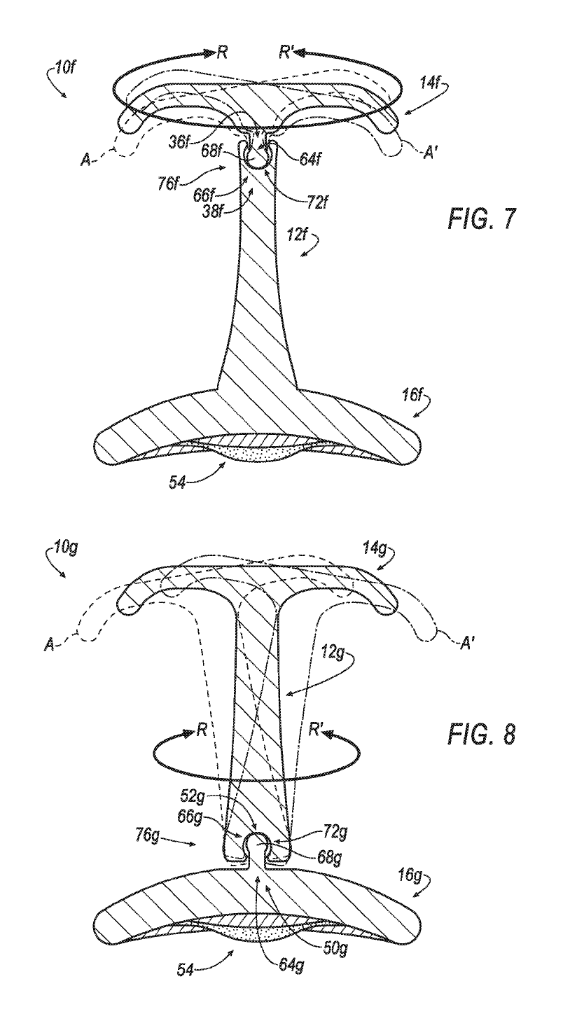

Although the hemostasis devices 10d, 10e provide the additional functionality associated with the permissive rotation, R/R′, it will be appreciated that the structure of the hemostasis devices 10d, 10e limits the rotated structure (i.e. the handle portion 14d/the handle portion 14e and stem portion 14e) to refrain from changing an angular pitch (i.e. an angular orientation) of the rotated structure relative the base portion 16d, 16e. Referring to FIGS. 7-9, hemostasis devices are shown generally at 10f, 10g, 10h in accordance with an embodiment of the invention. It will be appreciated that each of the hemostasis devices 10f, 10g, 10h include one or more ball-and-socket connections 76f, 76g that permits at least a portion of the hemostasis device 10f, 10g, 10h to be rotated, R/R′, and/or angularly pitched, A/A′, relative another portion of the hemostasis device 10f, 10g, 10h.

Referring to FIG. 7, an embodiment of the hemostasis device 10f is shown according to an embodiment of the invention. In an embodiment, the stem portion 12f and the base portion 16f may define a non-assembled, unitary structure that is connected to the handle portion 14f. In an embodiment, the connection surface 36f of the handle portion 14f may define, for example, a male portion 64f, and, the upper connection surface 38f of the stem portion 12f may define a female portion 66f. In an embodiment, the male portion 64f may be defined to include a ball portion 68f that is disposed into one a corresponding recess 72f defined by the female portion 66f. In an embodiment, the ball-and-socket connection 76f of the male portion 64f and the female portion 66f permits the handle 14f to rotate relative the stem portion 12f according to the direction of the arrow, R/R′, and, to be angularly pitched according to the direction of arrow, A/A′.

Referring to FIG. 8, an embodiment of a hemostasis device 10g is shown according to an embodiment of the invention. In an embodiment, the stem portion 12g and the handle portion 14g may define a non-assembled, unitary structure that is connected to the base portion 16g. In an embodiment, the connection surface 50g of the base portion 16g may define, for example, a male portion 64g, and, the lower connection surface 52g of the stem portion 12g may define, for example, a female portion 66g. In an embodiment, the male portion 64g may be defined to include a ball portion 68g that is disposed into one or more corresponding recesses 72g defined by the female portion 66g. In an embodiment, the ball-and-socket connection 76g of the male portion 64g and the female portion 66g permits the unitary stem portion 12g and handle portion 14g to rotate relative the stem portion 12g according to the direction of the arrow, R/R′, and, to be angularly pitched according to the direction of arrow, A/A′.

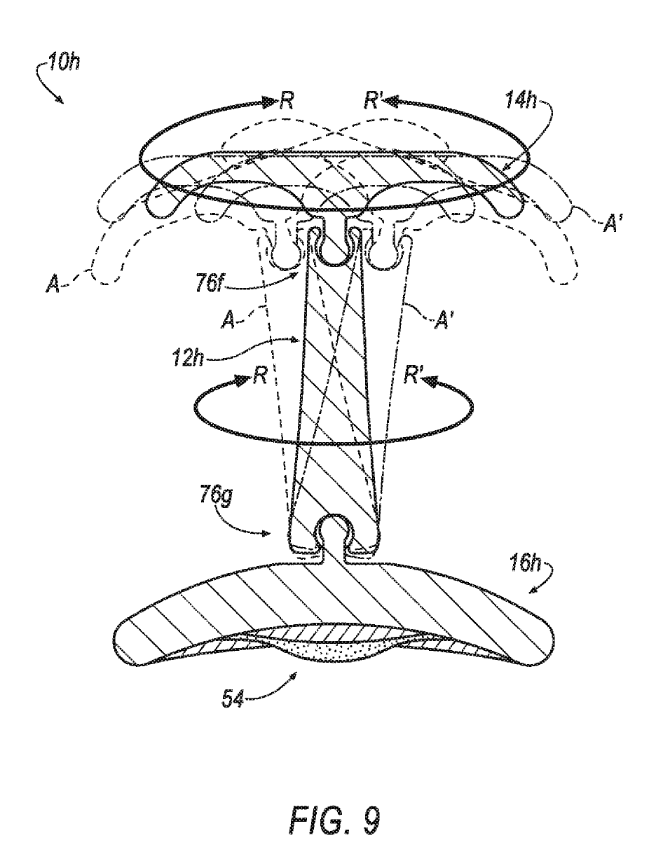

Referring to FIG. 9, an embodiment of a hemostasis device 10h is shown according to an embodiment of the invention. In an embodiment, the hemostasis device 10h does not include a unitary sub-substructure as shown in FIGS. 7 and 8, but rather, individual components that are connected in a substantially similar manner as that of the hemostasis devices 10a, 10b in FIGS. 1A and 1B.

In an embodiment, the handle portion 14h of the hemostasis device 10h is connected to the stem portion 12h by the ball-and-socket connection 76f described above, and, the base portion 16h of the hemostasis device 10h is connected to the stem portion 12h by the ball-and-socket connection 76g described above. Accordingly, when the base portion 16h is disposed against the patient, P, the handle portion 14h may be permitted to be rotated, R/R′, and angularly pitched, A/A′, relative the stem portion 12h as the stem portion 12h may be permitted to be rotated, R/R′, and angularly pitched, A/A′, relative the base portion 16h. It will be appreciated that although FIGS. 7-9 disclose ball-and-socket connections 76f, 76g, it will be appreciated that the invention is not limited to ball-and-socket connections 76f, 76g for permitting rotating/angular pitching movements and that any desirable connection may be provided to achieve the above-described movements.

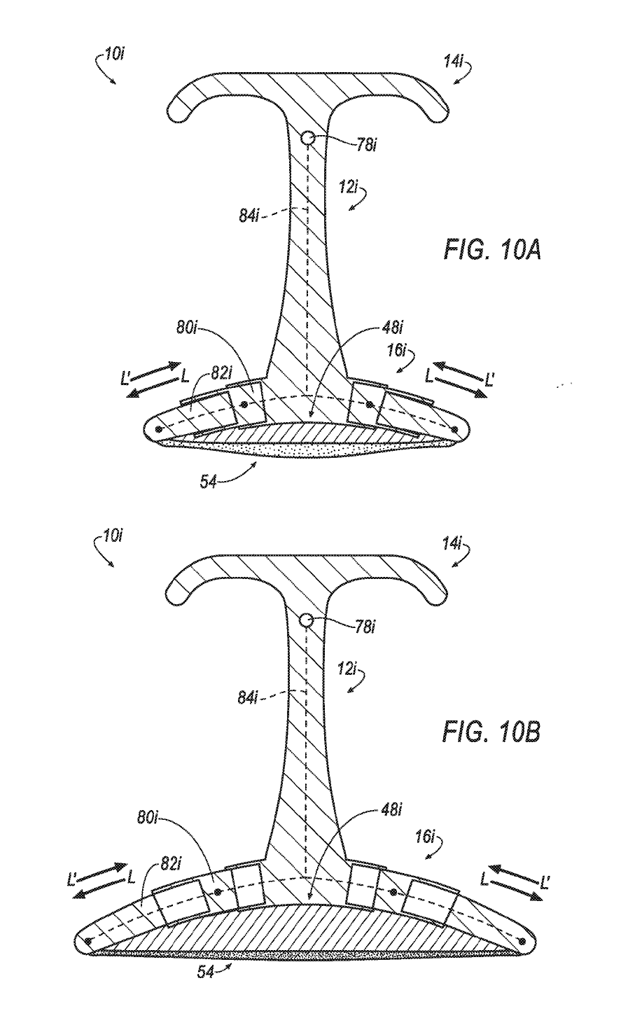

Referring to FIGS. 10A and 10B, in an embodiment, a hemostasis device 10i is shown according to an embodiment of the invention. In an embodiment, the hemostasis device 10i includes a stem portion 12i, a handle portion 14i and a base portion 16i. In an embodiment, the base portion 16i may provide an additional function that permits the base portion 16i to be laterally expanded or contracted according to the direction of arrows, L/L′, in order to selectively increase a surface area of the arcuate lower surface 48i of the base portion 16i.

In an embodiment, the hemostasis device 10i may include an actuator, which is shown generally at 78i, that is attached, to for example, the stem portion 12i. In an embodiment, the hemostasis device 10i may further include a plurality of telescoping, sections, which are shown generally at 80i, 82i, that comprise, or, are connected to the base portion 16i.

In an embodiment, the plurality of telescoping sections 80i, 82i, are laterally connected to the base portion 16i. In an embodiment, the actuator 78i may be connected to each of the plurality of telescoping sections 80i, 82i by a node 84i such that upon actuating the actuator 78i, the plurality of telescoping sections 80i, 82i may be laterally expanded, L, or laterally contacted, L′. In an embodiment, the node 84i may include any desirable mechanical, electro-mechanical, hydraulic or similar coupling device/mechanism that permits communicative control over the plurality of telescoping sections 80i, 82i upon de/actuating the actuator 78i.

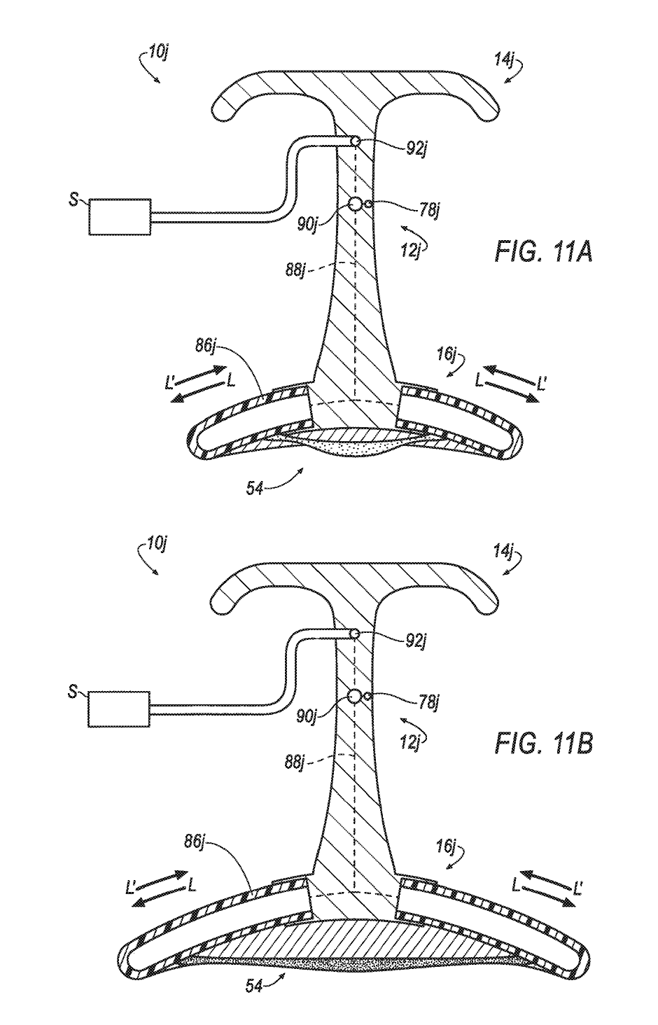

It will be appreciated that although the hemostasis device 10i discloses a plurality of telescoping sections 80i, 82i, the invention is not limited to a plurality of telescoping sections 80i, 82i. For example, in an alternative embodiment as seen in FIGS. 11A and 11B, a hemostasis device 10j may include a base portion 16j comprising a unitary, substantially hollowed, elastic material 86j rather than a plurality of telescoping sections 80i, 82i. In an embodiment, the unitary, substantially hollowed, elastic material 86j may be extended or retracted laterally, L/L′, by, for example, upon inflating the substantially hollowed, elastic material 86j.

In an embodiment, the hemostasis device 10j may include a fluid channel 88j having a normally-closed valve 90j. In an embodiment, the fluid channel 88j is in fluid communication with the hollowed, elastic material 86j. In an embodiment, the fluid channel 88j extends through the stem portion 12j. In an embodiment, the fluid channel 88j includes a fluid port 92j for permitting a source of pressurized fluid, S, to be connected to the hemostasis device 10j.

In an embodiment, an actuator 78j may be connected to the normally-closed valve 90j, and, upon actuating the actuator 78j, the normally-closed valve 90j may be moved from the normally-closed position to an open position to permit a fluid that is disposed in the source of pressurized fluid, S, to charge into the substantially hollowed, elastic material 86j for inflating and laterally-expanding, L, a surface area of the base portion 16j. After inflation of the hollowed, elastic material 86j, the actuator 78j may be released, thereby moving the valve 90j from the open position back to the normally-closed position, and then, the source of pressurized fluid, S, may be disconnected from the port 92j. Later, when it is desired to retract the base portion 16j to its pre-inflated orientation according to the direction of the arrow, L′, the actuator 78j may be actuated to move the normally-closed valve 90j from the normally-closed position back to the open position in order to permit the fluid contained within the hollowed, elastic material 86j to escape to atmosphere, thereby returning the hollowed, elastic material 86j to its pre-inflated state (as shown, e.g., in FIG. 11A).

Referring to FIGS. 12A-13, the hemostasis device 10a and a patient, P, is shown according to an embodiment. As shown in FIG. 12A, the hemostasis device 10a is shown being gripped by a user, U, such that the hemostasis device 10a is disposed proximate, for example, an abdominal wall, W, of the patient, P. Referring to FIG. 12B, the user, U, disposes the hemostasis device 10a adjacent the abdominal wall, W, of the patient, P, for compressing one or more of the femoral artery, FA, and femoral vein, FV, adjacent, for example, a bone, B (e.g. the femur), in order to achieve hemostasis.

By utilizing the hemostasis device 10a, the user, U, may direct a force vector, F, substantially perpendicularly relative to, for example, the abdominal wall, W. In an embodiment, as seen in FIG. 13, the force vector, F, may comprise, for example a plurality of externally-applied, substantially perpendicular force vector components, F₁-Fₙ, a substantially perpendicular, integrated force vector, F_{I}, and a plurality of substantially perpendicularly distributed force vector components, F_{D1}-F_{Dn}.

In an embodiment, as seen in FIG. 13, the force vector, F, originates proximate the handle 14 when the user, U, grips the handle 14 in order to act upon (e.g., apply an external force to) the hemostasis device 10a. Accordingly, the force vector, F, may include the plurality of externally-applied force components, F₁-Fₙ, that are channeled from the handle portion 14, proximate the second, cross-sectional geometry, G2, of the stem portion 12, which are subsequently collected proximate the narrowing, first cross-sectional geometry, G1, of the stem portion 12 to define the substantially perpendicular, integrated force vector, F_{I}. Once collected at the first cross-sectional geometry, G1, the substantially perpendicular, integrated force vector, F_{I}, is channeled toward the increasing, third cross-sectional geometry, G3, of the stem portion 12 such that the substantially perpendicular, integrated force vector, F_{I}, is distributed in, for example, a substantially perpendicularly even manner, as a plurality of distributed force vector components, F_{D1}-F_{Dn}, toward the base portion 16, which results in, for example, a substantially evenly-applied pressure, F_{P} (see, e.g., FIG. 12B), from the base portion 16 to one or more of a femoral artery, FA, and a femoral vein, FV, of the patient, P. It will be appreciated, however, that if, for example, the hemostasis device 10b having a variable point of contact is utilized, the plurality of distributed force vector components, F_{D1}-F_{Dn}, may be defined as a single distributed force vector component.

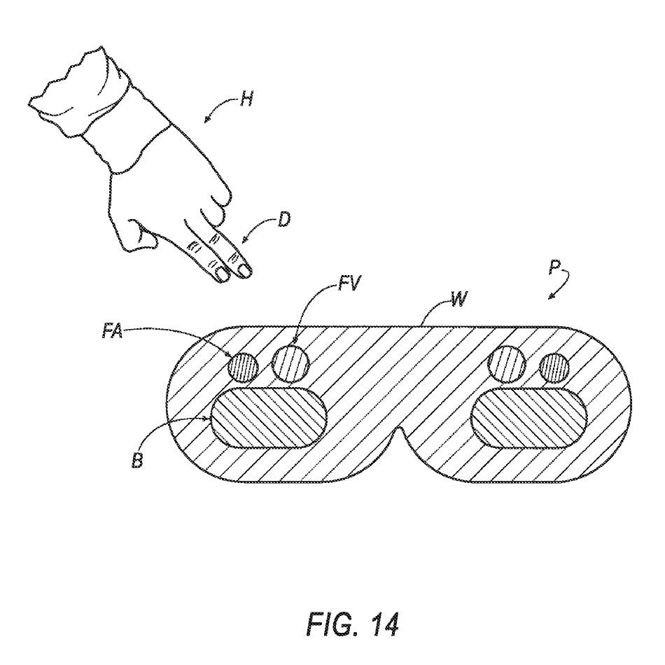

Further, it will be appreciated that the hemostasis device 10a may permit an amplification of an externally-applied force to a patient, P, when compared to conventional hemostasis methodologies (that is shown, for example, at FIG. 14). In an embodiment, it will be appreciated that plurality of externally-applied force components, F₁-Fₙ, may be amplified when compared to conventional methodologies due to the fact that the user, U, may utilize substantially all of his/her body weight arising from, for example, his/her upper torso rather than, for example, merely applying a force arising from one's digits, D (see, e.g., FIG. 14), which may be limited by, for example, a bent orientation of one's wrist. If, for example, one attempts to use the methodology shown in FIG. 14, and, for example, attempts to amplify the applied pressure to the patient by applying one's weight from their upper torso, it will be appreciated that a severe strain may be imparted to one's wrist; accordingly, although hemostasis of the patient, P, may be ultimately achieved, it may come at the expense of damage being sustained to, for example, one's wrist. As such, it will be appreciated that the hemostasis device 10a may overcome the difficulties associated with conventional hemostasis methodologies by providing an ergonomic device and method that may reduce and/or eliminate potential injures to a user, U.

Further, it will be appreciated that the applied pressure, Fp, arising from the hemostasis device 10a may be appropriately directed to one or more of the femoral artery, FA, and femoral vein, FV, thereby reducing or eliminating the likelihood of a non-perpendicular/non-channeled force vector, F, that would result in an asymmetrical pressure being applied to one or more of the femoral artery, FA, and femoral vein, FV. If, for example, an asymmetrical pressure was otherwise applied to one or more of the femoral artery, FA, and femoral vein, FV, by applying, for example, a conventional hemostasis methodology, hemostasis may not otherwise be achieved.

Further, it will be appreciated that the design of the hemostasis device 10a reduces the likelihood of fatigue of the user, U, due to the fact that the user, U, may utilize his/her body weight to apply the force vector, F, to, for example, the abdominal wall, W, of a patient, P. The hemostasis device 10a may overcome problems associated with conventional attempts to achieve hemostasis that include, for example, a prolonged strain of ligaments and/or muscles proximate a user's hand, wrist, forearm and shoulder area, which may otherwise contribute to hemostasis not being achieved.

Although the above described example in FIGS. 12A-13 discuss use of the hemostasis device 10a, it will be appreciated that the hemostasis devices 10b-10j function substantially the same as that of the hemostasis device 10a by applying pressure, Fp, to, for example, one or more of the femoral artery, FA, and femoral vein, FV, for achieving hemostasis. Further, it will be appreciated that one or more of the fixed, rotational and/or angular pitched movements R, R′/A/A′, associated with any of the hemostasis devices 10d-10h permits the user, U, to change his/her position relative the patient, P, to reduce fatigue. Further, it will it will be appreciated that one or more of the fixed, rotational and/or angular pitched movements R, R′/A, A′, associated with any of the hemostasis devices 10d-10h permits the user, U, to change his/her position relative the patient, P, if, for example, the patient, P, moves, which would result in the user, U, having to compensate for the orientation of the hemostasis device 10d-10h without having to remove the hemostasis device 10d-10h from the patient, P. Further, it will be appreciated that the lateral expansion, L, of the hemostasis devices 10i-10j permits the hemostasis devices 10i-10j to be utilized to cover and apply pressure, Fp, to a larger surface area of patients, P.

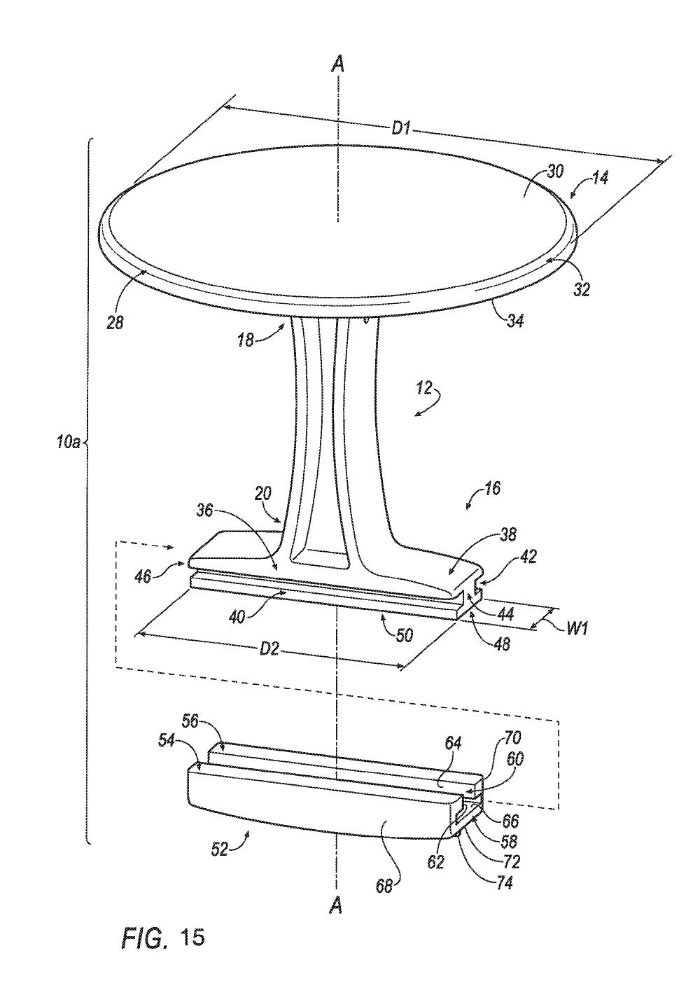

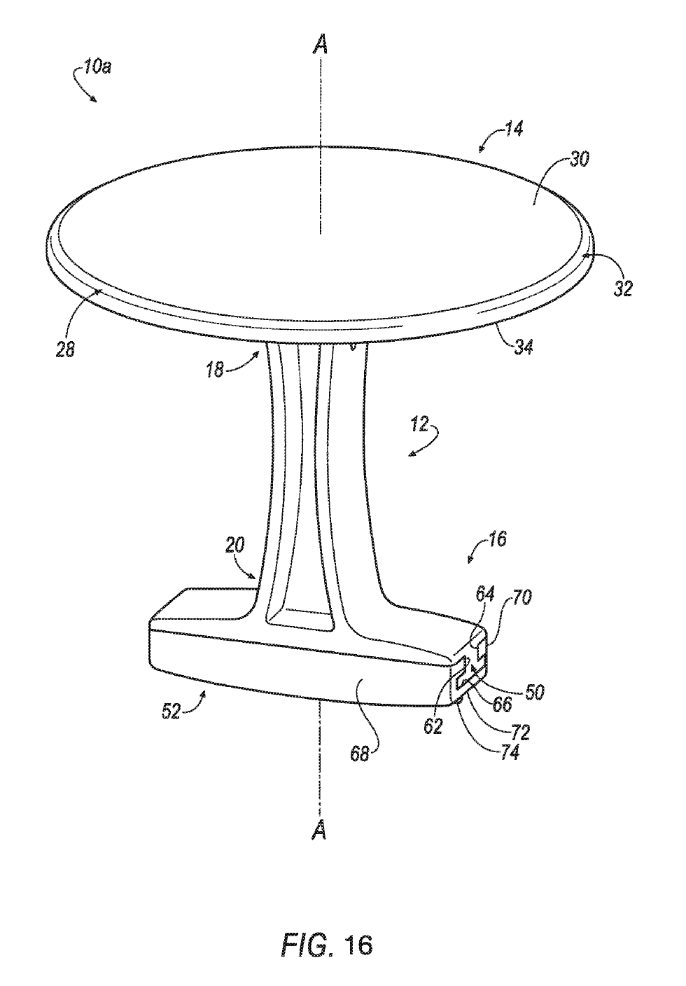

Referring to FIGS. 15-19, a hemostasis device is shown generally at 10a in accordance with an embodiment of the invention. In an embodiment, the hemostasis device 10a may include a centering pole/stem portion 12, a handle portion 14 and a base portion 16. In an embodiment, the stem portion 12 includes a first, top end 18 and a second, bottom end 20. In an embodiment, the handle portion 14 may be connected to the stem portion 12 at the first, top end 18, and, the base portion 16 may be connected to the stem portion 12 at the second, bottom end 20.

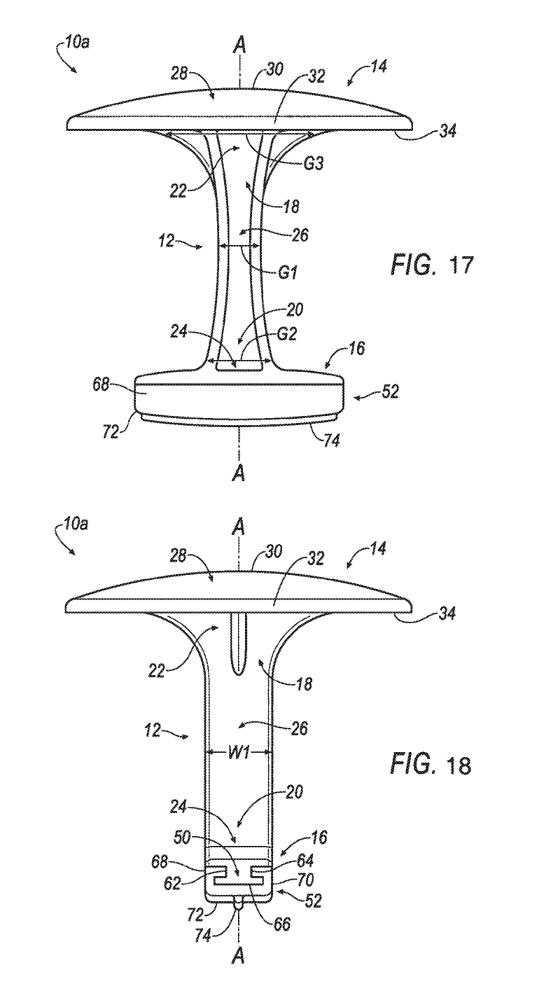

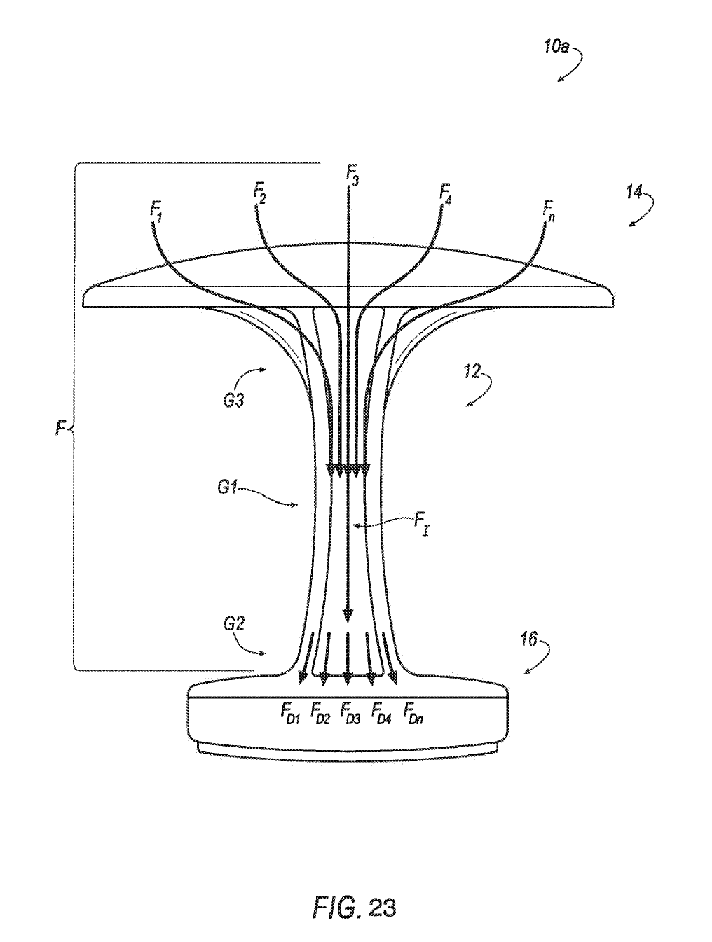

In an embodiment, referring to FIGS. 17 and 18, the stem portion 12 may define a head portion 22 proximate the first, top end 18, a shoulder portion 24 proximate the second, bottom end 20, and a neck portion 26 extending between the head portion 22 and the shoulder portion 24. In an embodiment, as seen in FIG. 17, the neck portion 26 may define a first, cross-sectional geometry, G1, that may be less than a second cross-sectional geometry, G2, defined by the shoulder portion 24, and, in an embodiment, the head portion 22 may define a third cross-sectional geometry, G3, that may be greater than the second cross-sectional geometry, G2, defined by the shoulder portion 24. The function of the geometries, G1-G3, of the stem portion 12 is described in greater detail in the foregoing disclosure at, for example, FIG. 23.

In an embodiment, as seen in FIG. 18, the stem portion 12 may include a substantially constant width, W 1, extending from the first, top end 18 to the second, bottom end 20. It will be appreciated however, that the width, W 1, may not be constant and may include, for example, any desirable dimension.

In an embodiment, as seen in FIGS. 15-18, the handle portion 14 may be defined by a substantially flat, disc-shaped body 28 having a diameter, D1. In an embodiment, the substantially flat, disc-shaped body 28 may include an upper, substantially arcuate, concave down surface 30, a circumferential arcuate side surface 32 and a lower, substantially planar surface 34.

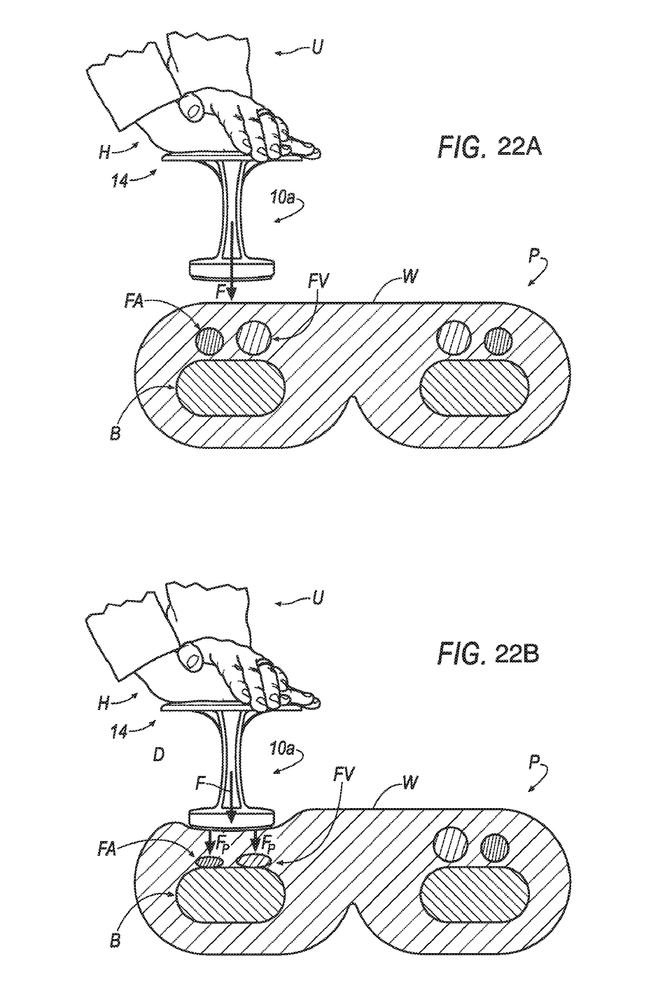

In an embodiment, the top end 18 of the stem portion 12 may integrally extend from an axial center of the lower surface 34 of the handle portion 14 relative a central axis, A-A. Functionally, in an embodiment as seen in FIGS. 22A-22B, a user, U, may locate a palm surface of one's hand, H, adjacent the upper surface 30 of the handle portion 14 such that a force, F, from the user, U, may be directed from the handle portion 14, through the stem portion 12 and out of the base portion 16.

Referring to FIG. 15, in an embodiment, the base portion 16 may be defined by a body 36 having a length, D2, that may be less than the diameter, D1, of the substantially flat, disc-shaped body 28. Further, in an embodiment, the body 36 may include a width that is substantially similar to the width, W 1, of the stem portion 12.

In an embodiment, the body 36 may include an upper surface 38, a first longitudinal side surface 40, a second longitudinal side surface 42, a first lateral side surface 44, a second lateral side surface 46 and a lower surface 48. In an embodiment, the bottom end 20 of the stem portion 12 may integrally extend from an axial center of the upper surface 38 relative the central axis, A-A.

In an embodiment, one or more of the longitudinal surface, lateral side surfaces and lower surface 40-48 may define a connection member 50. In an embodiment, as seen in FIGS. 15-16 and 18-20, the connection member 50 generally defines a T-shaped cross-sectional geometry. It will be appreciated, however, that the connection member 50 is not limited to include a particular cross-sectional geometry, and, as such, may include, for example, any desirable dimension, such as, for example, a dove-tail cross-sectional geometry 150 (see, e.g., FIG. 21) or the like.

Referring to FIGS. 15-19, in an embodiment, the hemostasis device 10a may further comprise a pad portion 52. Further, in an embodiment, as seen in FIG. 20, a hemostasis device 10b is shown including a pad portion 152, and, in an embodiment, as seen in FIG. 21, a hemostasis device 10c is shown including a pad portion 252.

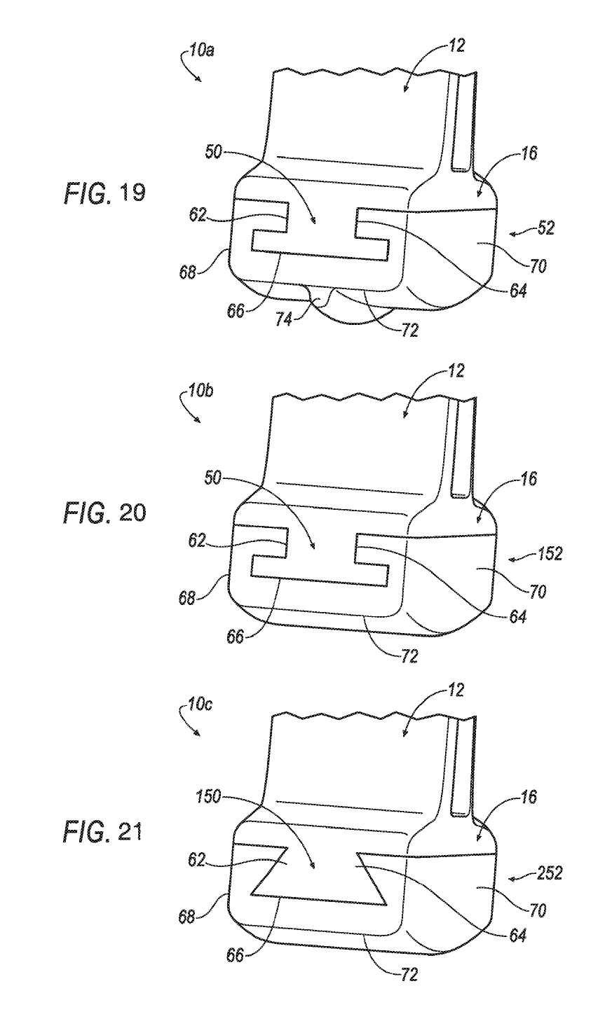

Referring to FIGS. 19 and 20, it will be appreciated that a pad portion 52, 152 may be selectively attached to the connection member 50 such that the user, U, may selectively-remove a pad portion 52, 152 from the connection member 50 in order to selectively-connect a particular pad portion 52, 152 to the connection member 50, if, for example, each of the pad portions 52, 152 include a similar geometry with respect to a passage or channel 60 (see, e.g., FIG. 15) that corresponds to the geometry of the connection member 50.

In an embodiment, as seen in FIG. 15, the pad portion 52 may include a first longitudinal portion 54, a second longitudinal portion 56 and an intermediate bridge portion 58 that connects the first longitudinal portion 54 to the second longitudinal portion 56. In an embodiment, the portions 54-58 generally define the pad portion 52 to include a substantially U-shaped cross-sectional geometry defining the longitudinal passage or channel 60.

In an embodiment, each of the first longitudinal portion 54, the second longitudinal portion 56 and the intermediate bridge portion 58 may include an inner longitudinal surface 62-66. In an embodiment, the inner longitudinal surfaces 62-66 collectively define the longitudinal passage or channel 60 to include a geometry that corresponds to that of the connection member 50.

In an embodiment, each of the first longitudinal portion 54, the second longitudinal portion 56 and the intermediate bridge portion 58 may include an outer longitudinal surface 68-72. In an embodiment, the outer longitudinal surfaces 68, 70 of the first longitudinal portion 54 and the second longitudinal portion 56 may define a substantially flat, outer surface whereas the outer longitudinal surface 72 of the intermediate bridge portion 58 may define a substantially flat, outer surface including a longitudinal bead 74 extending from the substantially flat, outer longitudinal surface 72.

In an embodiment, the longitudinal bead 74 may assist in providing the substantially flat, outer longitudinal surface 72 of the pad portion 52 with a frictional surface that assists in retaining the hemostasis device 10a at a desired position adjacent an abdominal wall, W, of a patient, P. Referring to FIG. 20, the pad portion 152 may be substantially similar to the pad portion 52 with the exception that the pad portion 152 does not include a bead extending from the outer longitudinal surface 72.

Referring to FIG. 21, the pad portion 252 may be substantially similar to the pad portion 52 with the exception that the pad portion 252 does not include a bead extending from the outer longitudinal surface 72. Further, the inner longitudinal surfaces 62-66 of the pad portion 252 collectively define the longitudinal passage or channel 60 to include a dove-tail geometry that corresponds to that of the dove-tail geometry of the connection member 150. Accordingly, it will be appreciated that one or more of the connection member 50, 150 and pad portion 52, 152, 252 are not limited to include any particular dimension, geometry or structure and may include any desirable configuration in order to provide a desired function, performance or the like for the hemostasis device 10a, 10b, 10c.

In an embodiment, it will be appreciated that the pad portion 52, 152, 252 may include any desirable material. In an embodiment, the pad portion 52, 152, 252 may include, for example, one or more materials that may be defined, for example, by a substantially soft consistency with a relatively constant density that is substantially independent to externally-applied forces. Further, in an embodiment, the pad portion 52, 152, 252 may further comprise a second material that may be defined by a substantially soft consistency with a variable density, which may depend on, for example, an externally-applied force.

Referring to FIGS. 22A-24, the hemostasis device 10a and a patient, P, is shown according to an embodiment. As shown in FIG. 22A, the hemostasis device 10a is shown with a portion of a palm of the user's hand, H, being disposed adjacent the upper, substantially arcuate, concave down surface 30 of the handle portion 14. In an embodiment, in order to retain the handle portion 14 adjacent the user's hand, H, the user, U, may locate their fingers/digits around the circumferential arcuate side surface 32 and the lower, substantially planar surface 34 in order to grip the handle portion 14.

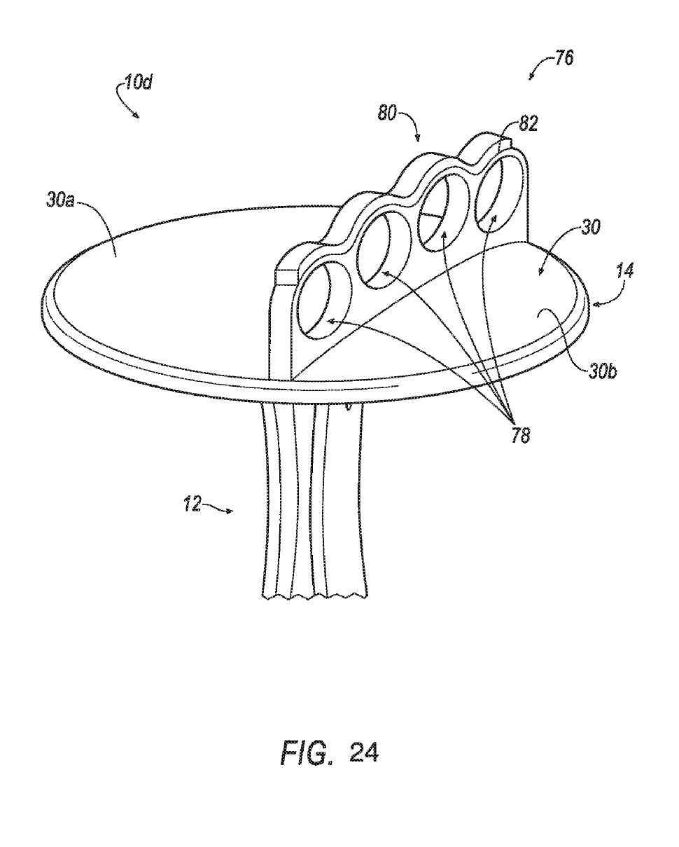

Referring to FIG. 24, in an alternative embodiment, a hemostasis device is shown generally at 10d and may include a digit-interfacing member 76 for interfacing one or more digits/fingers of a user's hand, H, with the hemostasis device 10d. In an embodiment, the digit-interfacing member 76 may be integral with and extend axially away from the upper, substantially arcuate, concave down surface 30 of the handle portion 14.

Accordingly, in an embodiment, the user, U, may be able to retain the handle portion 14 adjacent at least a portion of the user's hand, H, by inserting their fingers/digits through openings/passages 78 defined by the digit-interfacing member 76. Upon locating the fingers/digits through the openings/passage 78, the user, U, may place a palm surface of the hand, H, adjacent a first surface portion 30a of the upper, substantially arcuate, concave down surface 30 and the tips of the fingers/digits may be placed adjacent a second surface portion 30b of the upper, substantially arcuate, concave down surface 30.

In an embodiment, the digit-interfacing member 76 may also include a pad member 80 disposed adjacent an upper surface 82 of the digit-interfacing member 76. In an embodiment, the pad member 80 may provide a cushioning effect to the upper surface 82 of the digit-interfacing member 76 if, for example, the user, U, places a second hand over a first hand (as seen, for example, in FIGS. 22A, 22B) that is located adjacent the handle member 14, and, fingers/digits of which extend through the openings/passages 78.

Referring to FIG. 22A, the hemostasis device 10a may be disposed proximate, for example, an abdominal wall, W, of the patient, P. Referring to FIG. 22B, the user, U, disposes the hemostasis device 10a adjacent the abdominal wall, W, of the patient, P, for compressing one or more of the femoral artery, FA, and femoral vein; FV, adjacent, for example, a bone, B (e.g. the femur), in order to achieve hemostasis.

By utilizing the hemostasis device 10a, the user, U, may direct a force vector, F, substantially perpendicularly relative to, for example, the abdominal wall, W. In an embodiment, as seen in FIG. 23, the force vector, F, may comprise, for example a plurality of externally-applied, substantially perpendicular force vector components, F₁-Fₙ, a substantially perpendicular, integrated force vector, F_{I}, and a plurality of substantially perpendicularly distributed force vector components, F_{D1}-F_{Dn}.

In an embodiment, as seen in FIG. 23, the force vector, F, originates proximate the handle 14 when the user's hand, H, is interfaced with the handle 14 in order to act upon (e.g., apply an external force to) the hemostasis device 10a. Accordingly, the force vector, F, may include the plurality of externally-applied force components, F₁-Fₙ, that are channeled from the handle portion 14, proximate the third, cross-sectional geometry, G3, of the stem portion 12, which are subsequently collected proximate the narrowing, first cross-sectional geometry, G1, of the stem portion 12 to define the substantially perpendicular, integrated force vector, F_{I}. Once collected at the first cross-sectional geometry, G1, the substantially perpendicular, integrated force vector, F_{I}, is channeled toward the increasing, second cross-sectional geometry, G2, of the stem portion 12 such that the substantially perpendicular, integrated force vector, F_{I}, is distributed in, for example, a substantially perpendicularly even manner, as a plurality of distributed force vector components, F_{DI}-F_{Dn}, toward the base portion 16, which results in, for example, a substantially evenly-applied pressure, F_{P} (see, e.g., FIG. 22B), from the base portion 16 to one or more of a femoral artery, FA, and a femoral vein, FV, of the patient, P.

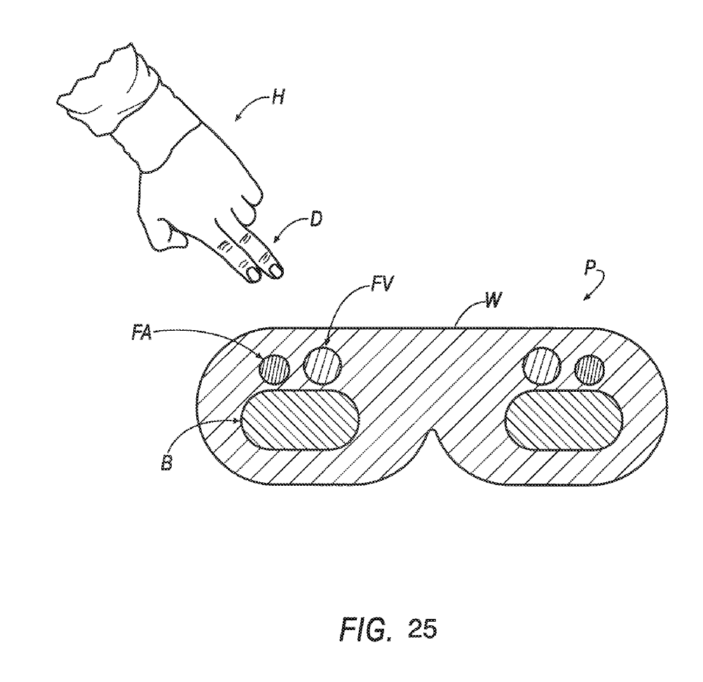

Further, it will be appreciated that the hemostasis device 10a may permit an amplification of an externally-applied force to a patient, P, when compared to conventional hemostasis methodologies (that is shown, for example, at FIG. 25). In an embodiment, it will be appreciated that plurality of externally-applied force components, F₁-Fₙ, may be amplified when compared to conventional methodologies due to the fact that the user, U, may utilize substantially all of his/her body weight arising from, for example, his/her upper torso rather than, for example, merely applying a force arising from one's digits, D (see, e.g., FIG. 25), which may be limited by, for example, a bent orientation of one's wrist. If, for example, one attempts to use the methodology shown in FIG. 25, and, for example, attempts to amplify the applied pressure to the patient by applying one's weight from their upper torso, it will be appreciated that a severe strain may be imparted to one's wrist; accordingly, although hemostasis of the patient, P, may be ultimately achieved, it may come at the expense of damage being sustained to, for example, one's wrist. As such, it will be appreciated that the hemostasis device 10a may overcome the difficulties associated with conventional hemostasis methodologies by providing an ergonomic device and method that may reduce and/or eliminate potential injures to a user, U.

Further, it will be appreciated that the applied pressure, Fp (see, e.g., FIG. 22B), arising from the hemostasis device 10a may be appropriately directed to one or more of the femoral artery, FA, and femoral vein, FV, thereby reducing or eliminating the likelihood of a non-perpendicular/non-channeled force vector, F, that would result in an asymmetrical pressure being applied to one or more of the femoral artery, FA, and femoral vein, FV. If, for example, an asymmetrical pressure was otherwise applied to one or more of the femoral artery, FA, and femoral vein, FV, by applying, for example, a conventional hemostasis methodology, hemostasis may not otherwise be achieved.

Further, it will be appreciated that the design of the hemostasis device 10a reduces the likelihood of fatigue of the user, U, due to the fact that the user, U, may utilize his/her body weight to apply the force vector, F, to, for example, the abdominal wall, W, of a patient, P. The hemostasis device 10a may overcome problems associated with conventional attempts to achieve hemostasis that include, for example, a prolonged strain of ligaments and/or muscles proximate a user's hand, wrist, forearm and shoulder area, which may otherwise contribute to hemostasis not being achieved. Although the above described example in FIGS. 22A-23 discuss use of the hemostasis device 10a, it will be appreciated that the hemostasis devices 10b-10d function substantially the same as that of the hemostasis device 10a by applying pressure, Fp, to, for example, one or more of the femoral artery, FA, and femoral vein, FV, for achieving hemostasis.

The present invention has been described with reference to certain exemplary embodiments thereof. However, it will be readily apparent to those skilled in the art that it is possible to embody the invention in specific forms other than those of the exemplary embodiments described above. This may be done without departing from the spirit of the invention. The exemplary embodiments are merely illustrative and should not be considered restrictive in any way. The scope of the invention is defined by the appended claims and their equivalents, rather than by the preceding description.

1.-33. (canceled)

34. A hemostasis device comprising:
a stem portion having a top end and a bottom end;
a handle portion coupled to the top end of the stem portion;
a base portion coupled to the bottom end of the stem portion, a length of the base portion being greater than a width of the base portion; and
a pad portion coupled to the base portion, the pad portion comprising a substantially flat bottom surface and a longitudinal bead extending from the bottom surface along at least part of a length of the pad portion.

35. The hemostasis device of claim 34, wherein the handle portion comprises a substantially disc-shaped body.

36. The hemostasis device of claim 35, wherein the substantially disc-shaped body comprises a substantially arcuate, concave down upper surface.

37. The hemostasis device of claim 34, wherein a width of the stem portion is substantially constant from the top end to the bottom end.

38. The hemostasis device of claim 34, wherein the stem portion is integrally formed with at least one of the handle portion and the base portion.

39. The hemostasis device of claim 34, wherein the pad portion is removably coupled to the base portion.

40. The hemostasis device of claim 34, wherein the base portion comprises a connection member configured to receive the pad portion.

41. A hemostasis device comprising:
a handle portion having an upper surface and a lower surface;
an elongate base portion having an upper surface and a lower surface, the base portion including a protrusion extending from the lower surface of the base portion along a length of the base portion; and
a stem portion extending from the lower surface of the handle portion to the upper surface of the base portion.

42. The hemostasis device of claim 41, wherein the stem portion extends from an axial center of the lower surface of the handle portion.

43. The hemostasis device of claim 41, wherein the stem portion extends to an axial center of the upper surface of the base portion.

44. The hemostasis device of claim 41, wherein the upper surface of the handle portion comprises a substantially arcuate, concave down surface.

45. The hemostasis device of claim 41, wherein the base portion further comprises a pad portion comprising the protrusion.

46. A hemostasis device comprising:
a stem having a proximal end and a distal end;
a handle, the proximal end of the stem comprising the handle; and
a base, the distal end of the stem comprising the base and the base comprising a protuberance extending distally and along a longitudinal axis of the base.

47. The hemostasis device of claim 46, wherein the base further comprises a pad.

48. The hemostasis device of claim 47, wherein the protuberance extends distally from a distal surface of the pad.

49. The hemostasis device of claim 47, wherein the base comprises a connection member configured to receive the pad.

50. The hemostasis device of claim 46, wherein the handle comprises a substantially disc-shaped body.

51. The hemostasis device of claim 50, wherein the substantially disc-shaped body comprises a substantially arcuate, concave down upper surface.

52. The hemostasis device of claim 46, wherein the stem is integrally formed with at least one of the handle and the base.

53. The hemostasis device of claim 46, wherein a width of the stem is substantially along the longitudinal axis of the base.
